(19) United States
(12) Patent Application Publication
(10) Pub. No.: US 2009/0197966 A1
(43) Pub. Date: Aug. 6, 2009

(54) Use of Hypothermia Inducing Drugs

(76) Inventors: Uno Jakob Weber, Bagsvaerd (DK); Jacob Gotfredsen, Copenhagen (DK)

Correspondence Address: NIXON & VANDERHYE, PC, 901 NORTH GLEBE ROAD, 11TH FLOOR, ARLINGTON, VA, 22203, US

(21) Appl. No.: 12/227,899

(22) PCT Filed: Oct. 4, 2007
(86) PCT No.: PCT/DK2007/050137

§ 371 Date: Apr. 16, 2009

(30) Foreign Application Priority Data
Oct. 4, 2006 (DK) PA 2006 01289
Mar. 6, 2007 (DK) PA 2007 00335

(51) Int. Cl. A61K 31/165; C07C 233/33; A61P 9/10

(52) U.S. Cl. 514/625; 564/192

(57) ABSTRACT

The present invention relates to the induction of hypothermia in humans in a predictable and dose responsive fashion by use of a pharmaceutical composition comprising a vanilloid receptor agonists, capsaicinoid or capsaicinoid-like agonist capable of inducing hypothermia, thereby benefiting patients suffering from illnesses characterized by tissue anoxia.

### FIELD OF INVENTION

The present invention relates to the use of compounds for the induction of hypothermia for the prophylaxis and treatment of ischemia. Ischemia is the lack of oxygenated blood flow to various body parts and may result from apoplexia, cardiac arrest and asphyxia.

### BACKGROUND OF INVENTION

Ischemia is the lack of oxygenated blood flow to various body parts and organs. Cerebral ischemia is an ischemic condition where the brain or parts of the brain do not receive enough blood flow to maintain normal neurological function. Cerebral ischemia can be the result of various serious diseases such as stroke and cardiac arrest, or the result of arterial obstruction such as strangulation. Severe or prolonged cerebral ischemia will result in unconsciousness, brain damage or death.

The neuroprotective efficacy of induced hypothermia following or during ischemia of the brain is evident in experimental animal models of stroke [1-11]. In humans, two trials conducted in cardiac arrest patients have shown improved neurological out-come of inducing hypothermia [12;13]. The therapeutic hypothermia did not increase the complication rate in these two trials and the use of induced hypothermia in comatose survivors of cardiac arrest is now recommended internationally [14].

Hypothermia counteracts ischemic brain damage by several mechanisms:
1. Ischemia induces opening of the blood-brain barrier, a process that seems to be very sensitive to brain temperature [15]. This is evident from studies of tracers and their migration across the blood-brain barrier, in which hypothermia attenuates extravasation several hours after ischemia [16] and prevents vasogenic oedema [17].
2. Reperfusion after brain ischemia results in the production of free radicals, which causes peroxidation and destruction of membrane lipids [18]. Hypothermia prevents the production of free radicals such as hydroxyl and nitric oxide during reperfusion after brain ischemia [19;20;24].
3. Amino acids, such as glutamate, aspartate, and glycine, act as excitotoxic neurotransmitters by over stimulation of neurons in the vicinity of ischemic damage, which causes further injury. Hypothermia lowers the release and may even cause a more rapid reuptake of these transmitters [21-23]. Release of excitotoxic neurotransmitters might also cause progressive neuronal death in the penumbra in stroke patients [22], and hypothermia after cerebral ischemia could attenuate this process.
4. During ischemia, cellular metabolism in the penumbra undergoes significant changes. As the neurons continue to fire, potassium ions flood into the extracellular space, calcium ions flow into the neurons leading to cytoskeletal degradation, and ATP concentrations fall as energy depletion continues [25]. Hypothermia reduces calcium influx and the subsequent breakdown of intracellular structures [26], improves potassium ion homoeostasis [27], and helps metabolic functions such as calcium or calmodulin-dependent protein kinase activity to recover [28;29].
5. By lowering of neutrophil and microglial activation after ischemia, hypothermia also has an anti-inflammatory effect [30;31].
6. Apoptosis and DNA changes are crucial stages in delayed neuronal death after transient cerebral ischemia [32]. Hypothermia directly inhibits apoptosis [33] and may also increase endogenous production of the anti-apoptotic protein Bcl-2 [34]. Hypothermia may even have effects at the DNA level: A slight lowering of brain temperature results in less DNA fragmentation [35] and less apoptosis [36].

Induction of hypothermia by lowering of the core temperature of the body has been attempted by mechanical cooling devices such as surface cooling and cooling using catheters placed in a large vessel. However, these mechanical inducers of hypothermia have been shown to have considerable unwanted side effects. These side effects include shivering, serious infections and lung puncture. Shivering causes an increased exertion of the heart of the patient, and this will in some cases result in ischemia of the heart and thereby increased morbidity and mortality.

The regulation of the core temperature of the body by a pharmaceutical composition comprising a compound capable of inducing hypothermia would not only solve the problem of reducing or preventing the effects of ischemia, such as tissue damaging effects, but also be relevant as a safer and less expensive alternative to the currently employed mechanical methods.

### SUMMARY OF INVENTION

The present invention relates to the induction of hypothermia in humans in a predictable and dose responsive fashion by use of a pharmaceutical composition comprising a compound capable of inducing hypothermia, thereby benefiting patients suffering from illnesses characterized by tissue anoxia. The inventors have found that such hypothermic effects can be obtained in humans as a result of compounds such as vanilloid receptor agonists, capsaicinoids or capsaicinoid-like agonists reaching and binding to vanilloid receptors.

Thus the present invention discloses the use of a compound for the induction of hypothermia for the preparation of a medicament for the treatment of ischemia in an individual.

It is also an aspect of the present invention to provide a medicament comprising a compound capable of inducing hypothermia in an individual.

A kit of parts comprising the medicament as herein disclosed is yet an aspect of the present invention.

Furthermore, the use of a compound according for the preparation of a medicament for obviating the induction of hypothermia in an individual, is an aspect of the present invention.

### DETAILED DESCRIPTION OF THE INVENTION

### Definitions

Agonist: A vanilloid receptor agonist is a vanilloid compound.
Antagonist: A vanilloid receptor antagonist is a substance capable of inhibiting the effect of a vanilloid receptor agonist.
Alcohol: A class of organic compounds containing one or more hydroxyl groups (OH). In this context a saturated or unsaturated, branched or unbranched hydrocarbon group sitting as a substituent on a larger molecule.
Alicyclic group: the term “alicyclic group” means a cyclic hydrocarbon group having properties resembling those of aliphatic groups.
Aliphatic group: in the context of the present invention, the term “aliphatic group” means a saturated or unsaturated linear or branched hydrocarbon group. This term is used to encompass alkyl, alkenyl, and alkynyl groups, for example.
Alkoxyl group: the term alkoxyl group or alkoxy covers an alkyl linked to a larger moiety by oxygen.
Alkyl group: the term “alkyl group” means a saturated linear or branched hydrocarbon group including, for example, methyl, ethyl, isopropyl, t-butyl, heptyl, dodecyl, octadecyl, amyl, 2-ethylhexyl, and the like.
Alkenyl group: the term “alkenyl group” means an unsaturated, linear or branched hydrocarbon group with one or more carbon-carbon double bonds, such as a vinyl group.
Alkynyl group: the term “alkynyl group” means an unsaturated, linear or branched hydrocarbon group with one or more carbon-carbon triple bonds.
Amphiphil: substance containing both polar, water-soluble and nonpolar, water-insoluble groups.
Aromatic Group: the term “aromatic group” or “aryl group” means a mono- or polycyclic aromatic hydrocarbon group.
Capsaicinoid: Compound capable of binding to a capsaicinoid receptor/vanilloid receptor and isolated from or identical to a compound isolated from an organism such as a plant or animal. In the present context any compound capable of binding a capsaicinoid receptor/vanilloid receptor. A capsaicinoid may also be referred to as a vanilloid receptor agonist.
Capsaicinoid-like: Compound capable of binding to a capsaicinoid receptor/vanilloid receptor and produced or synthesized chemically by standard techniques known in the art. In the present context any compound capable of binding a capsaicinoid receptor/vanilloid receptor. A capsaicinoid-like compound may also be referred to as a vanilloid receptor agonist.
Compound: A chemical substance formed from two or more elements, held together by chemical bonds, with a fixed ratio determining the composition. The elements lose their individual chemical properties and the compound has new properties. Herein a term covering all of the following: capsaicinoid, capsaicinoid-like and vanilloid receptor agonist.
Cyclic group: the term “cyclic group” means a closed ring hydrocarbon group that is classified as an alicyclic group, aromatic group, or heterocyclic group.
Cycloalkenyl: means a monovalent unsaturated carbocyclic radical consisting of one, two or three rings, of three to eight carbons per ring, which can optionally be substituted with one or two substituents selected from the group consisting of hydroxy, cyano, lower alkenyl, lower alkoxy, lower haloalkoxy, alkenylthio, halo, haloalkenyl, hydroxyalkenyl, nitro, alkoxycarbonenyl, amino, alkenylamino, alkenylsulfonyl, arylsulfonyl, alkenylaminosulfonyl, arylaminosulfonyl, alkylsulfonylamino, arylsulfonylamino, alkenylaminocarbonyl, arylaminocarbonyl, alkenylcarbonylamino and arylcarbonylamino.
Cycloalkyl: means a monovalent saturated carbocyclic radical consisting of one, two or three rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.
Cationic group: A chemical group capable of functioning as a proton donor when a compound comprising the chemical group is dissolved in a solvent, preferably when dissolved in water.
Form a ring: means that the atoms mentioned are connected through a bond when the ring structure is formed.
Group: (Moiety/substitution) as is well understood in this technical area, a large degree of substitution is not only tolerated, but is often advisable. Substitution is anticipated on the materials of the present invention. As a means of simplifying the discussion and recitation of certain terminology used throughout this application, the terms “group” and “moiety” are used to differentiate between chemical species that allow for substitution or that may be substituted and those that do not allow or may not be so substituted. Thus, when the term “group” is used to describe a chemical substituent, the described chemical material includes the unsubstituted group and that group with O, N, or S atoms, for example, in the chain as well as carbonyl groups or other conventional substitution. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, t-butyl, and the like. The same definitions apply to “alkenyl group” and “alkenyl moiety”; to “alkynyl group” and “alkynyl moiety”; to “cyclic group” and “cyclic moiety; to “alicyclic group” and “alicyclic moiety”; to “aromatic group” or “aryl group” and to “aromatic moiety” or “aryl moiety”; as well as to “heterocyclic group” and “heterocyclic moiety”.
Heterocyclic group: the term “heterocyclic group” means a closed ring hydrocarbon in which one or more of the atoms in the ring is an element other than carbon (e.g., nitrogen, oxygen, sulphur, etc.).
Heterocyclyl means a monovalent saturated cyclic radical, consisting of one to two rings, of three to eight atoms per ring, incorporating one or two ring heteroatoms (chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.
Heteroaryl means a monovalent aromatic cyclic radical having one to three rings, of four to eight atoms per ring, incorporating one or two heteroatoms (chosen from nitrogen, oxygen, or sulphur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.
Hypothermia: Lowering of the body temperature below normal level.
Ischemia: Restriction in blood supply with resultant dysfunction or damage of tissue.
Moieties of a particular compound cover group(s) or part(s) of said particular compound.
Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” encompass both the inactive drug and the active metabolite.
Substituted lower alkyl: means a lower alkyl having one to three substituents selected from the group consisting of hydroxyl, alkoxy, amino, amido, carboxyl, acyl, halogen, cyano, nitro and thiol.
Vanilloid receptor agonist: A capsaicinoid or capsaicinoid-like compound capable of binding a vanilloid receptor/capsaicinoid receptor.

The principle of the present invention is the use of vanilloid receptor agonists for induction of hypothermia for alleviating the effects of ischemia, such as tissue damaging effects of ischemia.

### Ischemia

Ischemia is the reduction or abolition of blood supply to a tissue. The associated deficiency of oxygen and nutrients may lead to cell death (necrosis) in areas of the affected tissue. The damage induced by the lack of oxygenated blood in the brain occurs in two stages. First cellular metabolism is arrested due to lack of oxygen and some cells and tissue will die within minutes as a consequence hereof. Secondly a cascade of processes such as apoptosis are initiated and continue up to 12 hours after the event that initially induced the ischemic state has been abolished. The tissue damaged by the second cascade can be crucial and cause greater harm to the individual than the primary damage happening within the first minutes of ischemia.

The current invention is aimed at correcting ischemia of the brain thereby minimizing the damage to the central nervous system. The invention does so by administering a drug to induce hypothermia in patients. The hypothermic effect is presumed to counteract ischemic damage by several mechanisms in the brain: Prevention of the blood-brain-barrier disruption that happens soon after ischemic onset that allows oedema formation from extravasation; Diminishing of the oxygen-based free-radical production; Reduction of the excitotoxic-neurotransmitter release that overstimulates neighboring neurons; Lowering of the metabolic rate and subsequent energy depletion; and anti-inflammatory action. Induction of hypothermia has a neuroprotective effect.

It is an object of the present invention to provide a compound capable of inducing hypothermia in a individual and further to provide the use of said compound for the production of a medicament for the treatment of ischemia in an individual.

Ischemia may occur under various circumstances; of special relevance to the present invention are the circumstances relating to cardiovascular diseases, asphyxia and traumatic brain injuries.

It is thus within the scope of the present invention to provide means for reducing the risk of ischemia as well as treating ischemia in an individual, under circumstances where ischemia is brought about by for example: cardiovascular diseases, asphyxia and traumatic brain injuries.

It is an aspect of the present invention to treat tissue damaging effects of ischemia.

### Cardiovascular Diseases

Cardiovascular disease is the most common cause of death and of physical as well as mental impairment in the developed world. A similar development is seen in the rest of the world as it emulates the lifestyle of the Western hemisphere with its fatty diets, lack of exercise and increasing average lifespan.

The main causes of death and disability among cardiovascular diseases are myocardial infarction, acute coronary syndrome, cardiac arrest and stroke, but many less common cardiovascular diseases may be equally detrimental to the individual affected. These less common diseases include among others arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

Each of the abovementioned diseases follow a course of events leading to ischemia, and are thus all of interest in relation to the present invention. Myocardial infarction (heart attack) is a result of an atherosclerotic plaque slowly building up in the inner lining of a coronary artery which then suddenly ruptures, partially or totally occluding the artery and preventing blood flow. Cardiac arrest is the abrupt cessation of normal circulation of the blood due to failure of the heart to contract effectively. Brain damage is likely to occur after 3-4 minutes without medical intervention, except in cases of hypothermia. Stroke is an acute neurological injury, lasting more than 24 hours, in which the blood supply to a part of the brain is interrupted, either by a clot in the artery or if the artery bursts. Arterial aneurism is a localized ballooning of an artery by more than 50% of the diameter of the vessel. Aneurysms most commonly occur in the arteries at the base of the brain and in the aorta. This bulge in an artery carries the risk of bursting and leading to internal hemorrhage. The larger an aneurysm becomes, the more likely it is to burst. Subarachnoid haemorrhage (SAH) is bleeding into the subarachnoid space surrounding the brain, i.e., the area between the arachnoid and the pia mater. It may arise due to trauma or spontaneously, and is a medical emergency, which can lead to death or severe disability even if recognized and treated in an early stage. Arteriosclerosis is a disease in which arterial walls harden over years or decades as a result of the formation of collagen and calcium deposits. Hypertension or high blood pressure is a medical condition wherein the blood pressure is chronically elevated. Hypercholesterolemia is the presence of high levels of cholesterol in the blood. It is a derangement that can contribute to many forms of disease, most notably cardiovascular disease. Arrhythmia is a group of conditions in which the muscle contraction of the heart is irregular or is faster or slower than normal. Some arrhythmias are life threatening medical emergencies that can cause cardiac arrest and sudden death. Cardiomegaly is a medical condition wherein the heart is enlarged. It can often be associated with other serious medical conditions. Cardiomyopathy is the deterioration of the function of the myocardium (i.e., the actual heart muscle). People with cardiomyopathy are at risk of arrhythmia and/or sudden cardiac death. Heart valve regurgitation, also known as heart valve insufficiency, is the abnormal leaking of blood through heart valves. Heart valve stenosis is a heart condition caused by the incomplete opening of a heart valve, typically the aortic valve or the mitral valve, impairing blood flow through the heart.

Each of the cardiovascular diseases mentioned, as well as others not mentioned, may cause ischemia of organs. This ischemia, whether of the brain, heart or other organs, may lead to death or impairment if not treated rapidly.

It is an object of the present invention to provide a compound for the production of a medicament for the treatment or prophylaxis of an individual suffering from or at risk of suffering from of ischemia due cardiovascular diseases such as, but not limited to: myocardial infarction, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

Preferably, the medicament is for the treatment or prophylaxis of ischemia due to cardiac arrest, myocardial infarction, stroke, arterial aneurisms, sub-arachnoid haemorrhage or angina pectoris.

All of the above-mentioned cardiovascular diseases require specific diagnostic tests and treatments. These tests and treatments, as specified for sudden cardiac arrest, stroke and heart attack in the below, may be carried out in conjunction with the treatment of the vanilloid receptor agonists defined in this patent.

Sudden cardiac arrest victims may be subjected to early CPR, early defibrillation and early advanced care. Further tests and treatments may include cardiac catheterization, electrophysiologic tests, coronary artery bypass surgery, balloon angioplasty or PTCA, antiarrhythmic medicine, implantable cardioverter/defibrillator, implantable pacemaker and heart transplant.

Depending on whether patients suffer an ischemic or hemorrhagic stroke, acute treatment may include clot-busters (e.g. tPA) or surgical intervention (e.g. aneurysm clipping, and endovascular procedures such as insertion of “coils”). Preventive treatment includes the administration of anticoagulants/anti-platelet. It may furthermore include carotid endarterectomy and angioplasty and/or stents.

Patients suffering a heart attack (myocardial infarction) may have one or several treatments and procedures done to survive and diagnose the condition: These include resuscitation (early CPR, early defibrillation, early advanced care), thrombolysis, coronary angioplasty (also known as Percutaneous Transluminal Coronary Angioplasty [PTCA], Percutaneous Coronary Interventions [PCI], Balloon Angioplasty and Coronary Artery Balloon Dilation), and coronary artery bypass graft surgery (CABG).

### Asphyxia

Asphyxia (suffocation) is a common cause of death and of physical as well as mental impairment in newborns, children and adults of all ages.

Asphyxia can be divided into perinatal asphyxia and non-perinatal asphyxia: Perinatal asphyxia is the medical condition resulting from deprivation of oxygen to a newborn infant long enough to cause apparent harm. It results most commonly from a drop in maternal blood pressure or interference during delivery with blood flow to the infant's brain. This can occur due to inadequate circulation or perfusion, impaired respiratory effort, or inadequate ventilation. Extreme degrees of asphyxia can cause cardiac arrest and death. Hypoxic damage can occur to most of the infant's organs, but brain damage is of most concern and perhaps the least likely to quickly and completely heal. In severe cases, an infant may survive, but with damage to the brain manifested as developmental delay and spasticity; Non-perinatal asphyxia is a condition of severely deficient supply of oxygen to the body that arises from being unable to breathe normally. Common causes hereof include drowning, strangulation and exposure to toxic gasses. Asphyxia causes generalized hypoxia, which primarily affects the tissues and organs most sensitive to hypoxia first, such as the brain, hence resulting in cerebral hypoxia. The absence of effective remedial action will very rapidly lead to unconsciousness, brain damage and death.

Each kind of asphyxia mentioned, as well as others not mentioned, may cause ischemia of organs and is thus an object of the present invention.

It is an aspect of the present invention to provide a compound for the treatment of an individual suffering from ischemia due to asphyxia such as: perinatal asphyxia and/or non-perinatal asphyxia.

Treatment by administration of the vanilloid receptor agonists defined in this patent may be carried out in conjunction with test and treatments of diseases and accidents including asphyxia (perinatal asphyxia, and non-perinatal asphyxia including, but not limited to, drowning, strangulation and exposure to toxic gasses). Such diseases and injuries may require early CPR, early defibrillation and both early and continued advanced care as well as other tests and treatments not specified.

### Traumatic Brain Injury

Traumatic brain injury (TBI) is a common cause of death and of physical as well as mental impairment throughout the world. TBI may result from accidents, be due to violence or be self-inflicted.

Traumatic brain injury, also called intracranial injury, or simply head injury, occurs when a sudden trauma causes brain damage. TBI can result from a closed head injury or a penetrating head injury. Parts of the brain that can be damaged include the cerebral hemispheres, cerebellum, and brain stem. Symptoms of a TBI can be mild, moderate, or severe, depending on the extent of the damage to the brain. Outcome can be anything from complete recovery to permanent disability or death. Ischemia is a significant factor contributing to the neurological damage frequently seen in patients suffering from TBI.

It is an aspect of the present invention to provide a compound for the treatment of an individual suffering from ischemia due to traumatic brain injury.

Treatment by administration of the vanilloid receptor agonists defined in this patent may also be carried out in conjunction with test and treatments in relation to traumatic head injury (closed head injury or penetrating head injury). Such injuries may require early CPR, early defibrillation and both early and continued advanced care as well as other tests and treatments not specified.

### Hypothermia

Hypothermia is the lowering of the core temperature of the body below normal level. Normal body temperature in an adult human measured rectally over 24 hours is 37 degree Celsius +/−0.6 degree Celsius and is thus variable between individuals, and over time within the individual. Hypothermia as a medical condition is usually defined as the effects seen on the body once the core temperature drops below 35 degree Celsius. It may become critical, if the body temperature falls below 32° C. In the present application hypothermia is defined as the lowering of the core body temperature below normal levels. This implies that any temperature below the normal core body temperature of the specific individual with its natural variations at the given point in time of the day, or period, herein is defined as being hypothermic. In particular, hypothermia is a temperature below 35.5° C., such as below 35° C., such as below 34.5° C., such as below 34.0° C.

Body temperature may be measured by a variety of means by mercury, electronic or plastic strip thermometers on different areas of the body such as the forehead, mouth, armpit, ear or rectum. It is presently understood, that the temperature referred to in the present application is the core body temperature, and that some of the above methods of measurement will indicate a different temperature than the core temperature.

It is of importance, that induction of hypothermia in an individual can follow a predictable course and be responsive to the dose in which the compound capable of inducing hypothermia is administered. The induction of the hypothermic condition may be rapid or slow depending on the situation of the individual in need of treatment. Also depending on the severity of the ischemic condition, it is of interest to provide a medicament for retaining the individual in the hypothermic state for variable durations of time. A single compound may be used depending on dosage within a range of temperatures or for the induction of hypothermia to a specific temperature. A combination of compounds may furthermore be used for an initial rapid decrease in core body temperature, and the subsequent maintenance of the reached temperature over a prolonged period. It is furthermore beneficial if the hypothermic state can be reversed in a controlled fashion either slowly or rapidly depending on the status of the individual.

It is thus an object of the present invention to provide a compound for the production of a medicament for the induction of hypothermia in an individual suffering from ischemia, wherein the compound is capable of inducing hypothermia to any range of temperatures between 37 and 31 degree Celsius, such as between 36.5 and 31.5 degree Celsius, such as between 36 and 32 degree Celsius, such as between 35.5 and 32.5 degree Celsius, such as between 35 and 33 degree Celsius, such as between 34.5 and 33.5 degree Celsius. The ranges may furthermore be between 37 and 34 degree Celsius, such as between 36.5 and 34.5 degrees, such as 36 and 35 degrees, alternatively between 34 and 31 degree, such as between 33.5 and 31.5 degree, such as 33 and 32 degree Celsius, alternatively between 36 and 33 degree or 35 and 32 degree Celsius. Preferably, the compound of the present is capable of inducing hypothermia in the range of between 36 to 32 degree Celsius, more preferably between 35 and 33 degree Celsius and most preferably between 34 and 32 degree Celsius.

It is also an object of the present invention to provide a compound capable of inducing hypothermia to a specific temperature such as 37 degree Celsius, 36.5 degree Celsius, 36 degree Celsius, 35.5 degree Celsius, 35 degree Celsius, 34.5 degree Celsius, 34 degree Celsius, 33.5 degree Celsius, 33 degree Celsius, 32.5 degree Celsius, 32 degree Celsius, 31.5 degree Celsius or 31 degree Celsius or most preferably, the compound of the present invention is capable of inducing hypothermia to any of the above specific temperatures within a range of +/−0.5 degree Celsius, the range thus being between +/−0.4 degree Celsius, such as between +/−0.3 degree Celsius, such as between +/−0.2 degree Celsius, or such as between +/−0.1 degree Celsius. The temperature range or specific temperature a given compound is capable of inducing is herein also referred to as the target temperature of the compound and/or the medicament comprising the compound.

### Vanilloid Receptor Agonists

Vanilloid receptor agonists are a group of chemicals which are capable of binding the Vanilloid Receptor 1 (VR1), also known as the Transient Receptor Potential Cation Channel, Subfamily V (TRPV1). The term vanilloid receptor agonist covers several groups of compounds including capsaicinoids and capsaicinoid-like compounds such as resiniferanoids and unsaturated dealdehydes. Before other types were discovered or synthesized, the term capsaicinoid referred to a group of secondary metabolites produced by plants belonging to the genus Capsicum, such as chili peppers. These compounds are the active components of chili peppers that produce the sensation of burning in the mouth, when ingested. Vanilloid receptor agonists may be divided into two classes based on origin and chemical composition:
1. Classical vanilloid receptor agonists, a group which includes naturally occurring compounds of chili peppers and other vanillamide derivatives.
2. Non-classical vanilloid receptor agonists, a group wherein the members generally do not comprise a vanillamide moiety.

Vanilloid receptor agonists are irritants for mammals, but have no effect on birds, indicating that the compounds may have evolved as a deterrent for herbivores. The various vanilloid receptor agonists have different pungencies as measured on the Scoville scale, with capsaicin and dihydrocapsaicin being the most potent of the classical vanilloid receptor agonists.

The use of capsaicin as a medicament is known both anecdotally and scientifically and is today widely used as a medicine to treat or relieve various ailments. The main clinical use of capsaicin is in the form of a topical ointment to successfully treat the pain of neuralgia such as caused by an infection with herpes zoster (shingles), diabetic neuropathy, rheumatism, fibromyalgia, various types of arthritis such as osteoarthritis or rheumatoid arthritis, and other forms of chronic pain. Capsaicin desensitizes nerves from pain by eliminating unneeded nerve cells through necrotic death and helps arthritis sufferers by lowering the levels of Decapeptide Substance P (DSP) in the synovial fluid of joints. Capsaicin breaks down DSP, which can destroy cartilage and also magnify the sensing of pain. Recently studies demonstrate that capsaicin is a potent anticancer agent. Capsaicin induces apoptosis in pancreatic and prostate cancer cells with no significant damage to the surrounding healthy cells. Capsaicin is known to affect NF-kB, and it is believed that it is through this protein, that capsaicin activates apoptotic proteins, leading to cell death. In another cancer related application, capsaicin containing candy provides significant pain relief of ulcers in the mouth which often develop in chemotherapy patients. Capsaicin pastes and balms are used to treat muscle and joint pains and medications containing vanilloid receptor agonists are used as an anti-inflammatory agents as the chemicals causes blood vessels to dilate. Capsaicin has furthermore been mentioned as a remedy to reduce serum cholesterol levels, relieve psoriasis and treat headaches, migraines and chronic sinus infections. Vanilloid receptor agonists have furthermore received interest as putatively neuroprotective substances. The neuroprotective effect is at least in part mediated by induction of hypothermia, as described in the below.

Any compound which can be defined as a vanilloid receptor agonist, capsaicinoid or capsaicinoid-like compound these being classical or non-classical vanilloid receptor agonist or compounds that otherwise bind vanilloid receptors falls within the scope of the present invention. The terms vanilloid receptor agonist, capsaicinoid or capsaicinoid-like are used interchangeably herein.

### Receptors

The sensation of burning pain elicited by vanilloid receptor agonists occurs by the selective activation of sensory neurons that convey information about noxious stimuli to the central nervous system. The selectivity is based primarily on the presence of the VR1/TRPV1 receptor, a nonselective cation channel to which the vanilloid receptor agonists, capsaicinoids and capsaicinoid-like compounds bind as agonists. TRPV1 is also activated by extracellular protons, and temperatures in the noxious range, suggesting that it functions as a transducer of painful thermal stimuli in vivo. By binding to the TRPV1 receptor, the vanilloid receptor agonists produce the same effect that excessive heat or abrasive damage would cause, explaining why the spiciness of capsaicin, without causing an actual chemical burn, is described as eliciting a burning sensation.

A number of different mechanisms have been proposed to account for the various effects of vanilloid receptor agonists. The vanilloid receptors, that are likely to be multimeric, non-selective cation channels composed of 6 transmembrane domains, will upon activation induce Ca²⁺ entry and subsequent release of sensory neuropeptides like calcitonin gene-related peptide (CGRP) and tachykinins (e.g. substance P) in addition to somatostatin. This in turn induces a range of actions, that may vary depending on tissue types, and include vasodilatation, plasma protein extravasation and immune cell accumulation in the innervated area as well as systemic anti-inflammatory and analgesic actions. To date it is not known exactly which mechanisms are responsible for the hypothermic effect of vanilloid receptor agonists, but it is speculated that the abovementioned reactions may play a part in it.

The TRPV1 receptor was originally named the Vanilloid Receptor 1 (VR1), as a vanilloid moiety constitutes an essential component of classical vanilloid receptor agonists, but has since changed name to TRPV1 due to the receptors extended homology with other TRP family members. TRPV1 is a member of the subfamily of TRPV receptors, which includes TRPV1-6. Several of these receptors render the cells expressing them sensitive to heat at different intervals, mechanic pressure and various compounds such as vanilloid receptor agonists, camphor and others. Combinations of the subtypes and various oligomers of the vanilloid receptors may account for the variations in physiological outcome observed following activation of receptors by different vanilloid receptor agonists. The oligomeric structure of the receptors increase the likelihood of discovering active substances with specificity for certain of the many organs that harbors vanilloid receptor agonists, thereby activating only parts of the vanilloid receptor system.

Vanilloid receptors are abundant in many organs of the body including neuronal tissues of the preoptic area, locus ceruleus, medial hypothalamus, reticular formation, and ventral thalamus. Vanilloid receptors have furthermore been found in non-neuronal “port of entry” tissues (e.g., skin, gut, airways, conjunctiva), the various cell types lining such tissues (i.e., keratinocytes, epithelia, endothelia, etc.), and also in various peripheral non-neuronal tissues of rodents and humans (e.g., kidney, lung, testis, pancreas, spleen, liver, stomach. skin, vascular smooth muscle, placenta, cornea, uterus, and bladder). The hypothalamus, with its abundance of vanilloid receptors, is of particular interest to this invention: It is a likely component in vanilloid receptor mediated hypothermia as it contains the temperature regulating centres of the CNS.

Receptors other than TRPV1 have been implicated in causing hypothermia upon binding of agonists hereto. Although some data suggests that capsaicin causes hypothermia by a cannabinoid receptor (CB1 and CB2) independent mechanism, there are hypothermia inducing vanilloid receptor agonists that are capable of interacting both with TRPV1 and especially CB2. Apart from TRPV3 mentioned above as a modulator of TRPV1 activity, the prokineticin receptors 1 and 2 (PKR1 and PKR2) have been shown to interact with TRPV1 and modulate its activity. Receptors to which the vanilloid receptor agonists, capsaicinoids and capsaicinoid-like compounds of the present invention may bind, includes, apart from TRPV1 and the other TRPV subfamily members, TRPV2, -3, -4, -5 and -6, CB1, CB2 and a third CB receptor, herein termed CB3, PKR1 and PKR2, GABA (gamma-aminobutyric acid) receptors, the NMDA (N-methyl-D-aspartate) receptor, the 5-HT(1A) receptor, also known as the serotonin receptor, the Delta opioid receptor (DOR). It is furthermore within the scope of the invention that the compounds of the invention may bind TRPV1 co-receptors. Compounds capable of binding any of the above-mentioned receptors thus fall within the scope of the present invention.

Preferably, a compound of the present invention binds TRPV1 and/or a TRPV1 associated receptor. The importance of the TRPV1 receptor for the induction of hypothermia in mammals is the dramatic hypothermic response that is elicited by systemic administration of capsaicin, a response which is absent in mice lacking the TRPV1 gene.

### Structure

The vanilloid receptor agonists of this application are, largely based on their structure, categorized as follows: classic vanilloid receptor agonists and non-classic vanilloid receptor agonists. Compounds belonging to any of these categories fall within the scope of the present invention.

It is within the scope of the invention that the compounds of the invention are capable of inducing hypothermia in an individual.

It is furthermore within the scope of the invention that the compounds of the invention are capable of binding the TRPV1 receptor and/or a receptor associated herewith.

Accordingly, in the broadest aspect the present invention concerns the use of a compound comprising a structure of one of the general formulas illustrated in the below. In these illustrations R is a chemical bond or a chemical moiety as defined in the above. R may be any moiety substituted any amount of times according to the following non-limiting list, whereby R is: C, H, S, N, O, optionally substituted one or more times with C, H, S, N, O, B, P, OH, CHO, hydrogen, alkoxy, alkyl, alkenyl, alkynyl, phenyl, diphenyl, benzyl, amine (NH), halogen, substituted lower alkyl, alkenyl, aryl, heterocyclic group, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl or phosphate, optionally further substituted one or more times with C, S, N, O, P, OH, H, phenyl, amine (NH), halogen, alkoxy, substituted lower alkyl or alkyl, alkenyl or alkynyl such as (C₁-C_{V}), acetyl, sulfonyl, phenyl, cycloalkyl, cycloalkenyl, heterocyclyl or heterocyclic group any of which may or may not be branched or be further substituted one or more times with C, O, P, methyl, dimethyl, alkyl or alkenyl such as (C₁-C_{V}), alkoxy, phenyl, sulphate, phosphate, halogen or further substituted by fluoride, sulphate, phosphate, methyl, dimethyl, aryl, heterocyclyl, heteroaryl, aryl-(C₁₋₄)-alkyl, heteroaryl-(C₁₋₄)-alkyl, heterocyclyl-(C₁₋₄)alkyl, cycloalkylalkyl, dicycloalkyl, tricycloalkyl, cycloalkenyl, alkoxy, carboxy, halogen, trifluoromethyl, cyano, amino, nitro, halogen or alcohol, and further substituted at least once with O, OH, methyl, alkenyl, cycloalkyl, heterocycloalkyl, cycloalkenyl, dimethyl or phenyl further substituted with alkyl, alkenyl, cycloalkyl, cycloalkenyl, heterocyclyl and wherein _{V} is an integer of from 1 to 30, and of which any of the mentioned substituents capable thereof may form a ring with another R. R may furthermore be a chemical bond, or a pharmaceutically acceptable addition salt or hydrate of any of the above.

For each general formula, a more specific choice of substituent for a given R is listed along with along with a preferred and a more preferred list of substituent groups.

The present invention concerns the use of a compound such as a classic or non-classic vanilloid receptor agonist comprising the general formula (I):

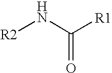

wherein R1 is selected from the group of: C, S, N, O, optionally substituted with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, S, N, O, P, OH, methoxy, ethoxy, acetyl, alkoxy, alkyl, alkenyl, alkynyl, sulfonyl or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted with C, O, P, H, OH, phosphate, alkyl, alkenyl, alkynyl any of which may be (C₁-C_{V}), or phenyl, any of which may be substituted with O, OH, methyl, dimethyl, alkyl, alkenyl, cycloalkyl, heterocycloalkyl, cycloalkenyl, phenyl, methoxy, ethoxy, alkoxy or phosphate, any of which may be further substituted with methyl, ethyl or phenyl and more preferably is C substituted with alkyl, alkenyl, any of which may be (C₄-C_{W}) any of which may be further substituted with O, OH, methoxy, ethoxy or methyl any of which may be further substituted with methyl, ethyl, or phenyl, wherein _{V} is an integer of from 1 to 30 and _{W} is an integer of from 5 to 18, and;
wherein R2 is selected from the group of: C, S, N, O, optionally substituted one or more times with C, S, N, O, P, OH, hydrogen, alkoxy, alkyl, alkenyl, alkynyl, phenyl, diphenyl, benzyl, amine (NH), halogen, substituted lower alkyl, alkenyl, aryl, heterocycloalkyl, heteroaryl, aryl-(C₁₋₄)-alkyl, heteroaryl-(C₁₋₄)-alkyl, heterocyclyl-(C₁₋₄)alkyl, cycloalkylalkyl, cycloalkyl, cycloalkenyl or phosphate, optionally further substituted one or more times with C, S, N, O, P, OH, H, COOH, phenyl, amine (NH), halogen, alkoxy, substituted lower alkyl, alkyl or alkenyl such as (C₁-C_{V}), cycloalkenyl, sulphate, phosphate, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, dicycloalkyl, tricycloalkyl, cycloalkenyl, alkoxy, carboxy, halogen, cyano, amino, nitro, or alcohol, any of which may be further substituted one or more times with OH, methyl, dimethyl, alkyl or alkenyl such as (C₁-C_{V}), alkoxy, phenyl, sulphate, phosphate, aryl, heteroaryl, carboxy, amino, nitro, alcohol or halogen and preferably is C, substituted one or more times with C, N, O, P, OH, hydrogen, alkoxy, alkyl, alkenyl, amine (NH), halogen, substituted lower alkyl, alkenyl, aryl, cycloalkyl, cycloalkenyl or phosphate, optionally further substituted one or more times with C, N, O, OH, COOH, hydrogen, amine (NH), halogen, alkoxy, substituted lower alkyl or alkenyl such as (C₁-C_{X}), phosphate, cycloalkenyl, alkoxy, carboxy or halogen, any of which may be further substituted one or more times with OH, methyl, dimethyl, alkyl or alkenyl such as (C₁-C_{X}), alkoxy, phenyl, sulphate, phosphate, carboxy or halogen and more preferably is C substituted with either: cycloalkenyl further substituted at least twice with any of OH or methoxy, or: lower alkyl such as (C₁-C₂) at least once and further substituted one or more times with OH, COOH, Chloride, methyl or cycloalkenyl, optionally further substituted one or more times with OH or methoxy and wherein _{V} is an integer of from 1 to 30 and _{X} is an integer of from 1 to 5.

Preferably, the present invention concerns the use of a compound comprising the general formula (I) wherein R1 is C substituted with C, O, P, H, OH, phosphate, alkyl, alkenyl, alkynyl any of which may be (C₁-C_{V}), or phenyl, any of which may be further substituted one or more times with O, OH, methyl, dimethyl, alkyl, alkenyl, cycloalkyl, heterocycloalkyl, cycloalkenyl, acetyl, phenyl, methoxy, ethoxy, alkoxy or phosphate, any of which may be further substituted with methyl, ethyl or phenyl and R2 is C, substituted one or more times with C, N, O, P, OH, hydrogen, alkoxy, alkyl, alkenyl, amine (NH), halogen, substituted lower alkyl, alkenyl, aryl, cycloalkyl, cycloalkenyl or phosphate, optionally further substituted one or more times with C, N, O, OH, COOH, hydrogen, amine (NH), halogen, alkoxy, substituted lower alkyl or alkenyl such as (C₁-C_{X}), phosphate, cycloalkenyl, alkoxy, carboxy or halogen, any of which may be further substituted one or more times with OH, methyl, dimethyl, alkyl or alkenyl such as (C₁-C_{X}), alkoxy, phenyl, sulphate, phosphate, carboxy or halogen and wherein _{V} is an integer of from 1 to 30 wherein _{X} is an integer of from 1 to 5.

Most preferably, the present invention concerns the use of a compound comprising the general formula (I) wherein R1 is C substituted with alkyl, alkenyl, any of which may be (C₄-C_{W}) any of which may be further substituted one or more times with O, OH, acetyl, methoxy, ethoxy or methyl any of which may be further substituted one or more times with methyl, ethyl, cycloalkenyl or phenyl and _{W} is an integer of from 5 to 18, and R2 is C substituted with either: cycloalkenyl further substituted at least twice with any of OH or methoxy, or: lower alkyl such as (C₁-C₂) at least once and further substituted one or more times with OH, COOH, Chloride, methyl or cycloalkenyl, optionally further substituted one or more times with OH or methoxy.

The present invention furthermore concerns the use of a compound such as a non-classic vanilloid receptor agonist comprising the general formula (II):

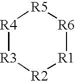

wherein R1 is selected from the group of: C, S, N, O, optionally substituted with C, S, N, O, B, P, OH, hydrogen, alkyl, alkenyl, alkynyl, any of which may or may not be branched or comprise substituents such as phosphate, cycloalkyl, heterocyclyl, heterocyclic group, cycloalkenyl, methyl, ethyl, dimethyl, or may be further substituted one or more times with C, S, N, O, P, OH, methoxy, ethoxy, acetyl, alkoxy, alkyl, alkenyl, alkynyl, sulfonyl or phenyl cycloalkyl, cycloalkenyl, heterocyclyl, heterocyclic group any of which may or may not be branched or comprise substituents such as C, O, H, OH, methyl, ethyl, alkyl, alkenyl, alkynyl, alkoxy, phosphate, further substituted at least once with O, OH, methyl, alkenyl, cycloalkyl, heterocycloalkyl, cycloalkenyl, dimethyl or phenyl further substituted with alkyl, alkenyl, cycloalkyl, cycloalkenyl, heterocyclyl and preferably is C substituted with C, N, O, B, P, OH, hydrogen, alkyl, alkenyl, alkynyl, any of which may or may not be branched or comprise substituents such as phosphate, cycloalkyl, heterocyclyl, heterocyclic group, cycloalkenyl, methyl, ethyl, dimethyl, or may be further substituted one or more times with C, N, O, P, OH, methoxy, ethoxy, acetyl, alkoxy, alkyl, alkenyl, cycloalkyl, cycloalkenyl, heterocyclyl, heterocyclic group any of which may or may not be branched or comprise substituents such as C, O, H, OH, methyl, ethyl, alkyl, alkenyl, alkoxy, phosphate, further substituted at least once with O, OH, methyl, alkenyl, cycloalkyl, heterocycloalkyl, cycloalkenyl, further substituted with alkyl, alkenyl, cycloalkyl, cycloalkenyl, heterocyclyl and more preferably is C substituted with alkyl or alkenyl which may be (C4-C_{W}), B, or heterocyclyl forming a ring with R6 thus comprising a pyrrolidine further substituted one or more times with O, methyl, alicyclic groups attached to one another, these being saturated or unsaturated or a heterocyclic group comprising said B, N and O, and being further substituted at least once with O, methyl or C further substituted at least once with cycloalkenyl, cycloalkyl, heterocyclyl further substituted at least once with O, OH, methyl, alkenyl, further substituted with lower alkyl and cycloalkenyl; wherein any of the above may form at least one bond with any of R2, R3, R4, R5 and/or R6, and w is an integer of from 5 to 18, and;
wherein R2 is selected from the group of C, S, N, O, optionally substituted at least once with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, N, O, P, OH, methoxy, ethoxy, acetyl, alkoxy, alkyl, alkenyl, alkynyl, or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted with C, O, P, H, OH, phosphate, alkyl, alkenyl, alkynyl any of which may be (C₁-C_{V}), or phenyl, any of which may be substituted with O, OH, methyl, dimethyl, alkyl, alkenyl, cycloalkyl, heterocycloalkyl, cycloalkenyl, phenyl, methoxy, ethoxy, alkoxy or phosphate, and more preferably is C substituted at least once with OH or hydrogen, and wherein any of the above may form at least one bond with any of R1, and/or R3, and wherein _{V} is an integer of from 1 to 30; and
wherein R3 is selected from the group of C, S, N, O, optionally substituted with C, S, N, O, B, P, OH, hydrogen, methoxy, ethoxy, acetyl, alkoxy, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, S, N, O, P, OH, methoxy, ethoxy, acetyl, alkoxy, alkyl, alkenyl, alkynyl, or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted with C, O, P, H, OH, phosphate, acetyl, alkoxy, alkyl, alkenyl, any of which may be (C₁-C_{V}), or phenyl, optionally substituted with O, OH, methyl, dimethyl, alkyl, alkenyl, phenyl, methoxy, ethoxy, alkoxy or phosphate, and more preferably is C substituted with OH, methoxy, lower alkyl (C1-C_{Y}) and may at any point form a ring with R4, and wherein any of the above may form at least one bond with any of R2, and/or R4, and wherein _{V} is an integer of from 1 to 30, and _{Y} is an integer of 2 or 3; and
wherein R4 is selected from the group of C, S, N, O, optionally substituted with C, S, N, O, B, P, OH, hydrogen, methoxy, ethoxy, acetyl, alkoxy, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, S, N, O, P, OH, methoxy, ethoxy, acetyl, alkoxy, alkyl, alkenyl, alkynyl, or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted with C, O, P, H, OH, phosphate, acetyl, alkoxy, alkyl, alkenyl, any of which may be (C₁-C_{V}), or phenyl, optionally substituted with O, OH, methyl, dimethyl, alkyl, alkenyl, phenyl, methoxy, ethoxy, alkoxy or phosphate, and more preferably is C substituted with OH, methoxy, lower alkyl (C1-C_{Y}) and may at any point form a ring with R4, and wherein any of the above may form at least one bond with any of R2, and/or R4, and wherein _{X} is an integer of from 1 to 30; and _{Y} is an integer of 2 or 3; and
wherein R5 is selected from the group of C, S, N, O, optionally substituted with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, S, N, O, P, OH, CHO, hydrogen, methoxy, ethoxy, acetyl, alkoxy, alkyl, alkenyl, alkynyl, sulfonyl or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted at least once with C, O, P, H, OH, CHO, phosphate, alkyl, alkenyl, any of which may be (C₁-C_{V}), or phenyl, any of which may be substituted with O, OH, methyl, dimethyl, alkyl, alkenyl, methoxy, ethoxy, alkoxy or phosphate, and more preferably is C substituted one or more times with hydrogen, OH, CHO or methyl which may form a ring with R6 wherein _{V} is an integer of from 1 to 30, and;
wherein R6 is selected from the group of C, S, N, O, optionally substituted with C, S, N, O, B, P, OH, CHO, hydrogen, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, N, O, P, OH, methoxy, ethoxy, acetyl, alkoxy, alkyl, alkenyl, alkynyl, or phenyl any of which may or may not be branched or comprise substituents such as hydrogen, methyl, ethyl, alkyl, alkenyl, alkynyl, alkoxy, phosphate, dimethyl or phenyl and preferably is C substituted with C, O, P, H, OH, CHO, phosphate, alkyl, alkenyl, any of which may be (C₁-C_{V}), or phenyl, any of which may be substituted with O, OH, methyl, dimethyl, alkyl, alkenyl, methoxy, ethoxy, alkoxy or phosphate, and more preferably is C substituted one or more times with hydrogen, CHO, lower alkyl (C1-C_{Y}) or methyl, which may form at least one bond with R5, or be at least one bond forming a ring with R1, wherein the at least one bond between any of R1, R2, R3, R4 and/or R5 may be single or double bonds and wherein _{V} is an integer of from 1 to 30; and _{Y} is an integer of 2 or 3.

Preferably, the present invention concerns the use of a compound comprising the general formula (I) wherein R1 is C substituted with C, N, O, B, P, OH, hydrogen, alkyl, alkenyl, alkynyl, any of which may or may not be branched or comprise substituents such as phosphate, cycloalkyl, heterocyclyl, heterocyclic group, cycloalkenyl, methyl, ethyl, dimethyl, or may be further substituted one or more times with C, N, O, P, OH, methoxy, ethoxy, acetyl, alkoxy, alkyl, alkenyl, cycloalkyl, cycloalkenyl, heterocyclyl, heterocyclic group any of which may or may not be branched or comprise substituents such as C, O, H, OH, methyl, ethyl, alkyl, alkenyl, alkoxy, phosphate, further substituted at least once with O, OH, methyl, alkenyl, cycloalkyl, heterocycloalkyl, cycloalkenyl, further substituted with alkyl, alkenyl, cycloalkyl, cycloalkenyl, heterocyclyl any of the above of which may form bonds with R2, and/or R6, and R2 preferably is C substituted with C, O, P, H, OH, phosphate, alkyl, alkenyl, alkynyl any of which may be (C₁-C_{V}), or phenyl, any of which may be substituted with O, OH, methyl, dimethyl, alkyl, alkenyl, cycloalkyl, heterocycloalkyl, cycloalkenyl, phenyl, methoxy, ethoxy, alkoxy or phosphate, and R3 preferably is C substituted with C, O, P, H, OH, phosphate, acetyl, alkoxy, alkyl, alkenyl, any of which may be (C₁-C_{V}), or phenyl, any of which may be substituted with O, OH, methyl, dimethyl, alkyl, alkenyl, phenyl, methoxy, ethoxy, alkoxy or phosphate, and R4 preferably is C substituted with C, O, P, H, OH, phosphate, acetyl, alkoxy, alkyl, alkenyl, any of which may be (C₁-C_{V}), or phenyl, optionally substituted with O, OH, methyl, dimethyl, alkyl, alkenyl, phenyl, methoxy, ethoxy, alkoxy or phosphate, and R5 preferably is C substituted at least once with C, O, P, H, OH, CHO, phosphate, alkyl, alkenyl, any of which may be (C₁-C_{V}), or phenyl, any of which may be substituted with O, OH, methyl, dimethyl, alkyl, alkenyl, methoxy, ethoxy, alkoxy or phosphate, and R6 preferably is C substituted with C, O, P, H, OH, CHO, phosphate, alkyl, alkenyl, any of which may be (C₁-C_{V}), or phenyl, any of which may be substituted with O, OH, methyl, dimethyl, alkyl, alkenyl, methoxy, ethoxy, alkoxy or phosphate, and wherein _{V} is an integer of from 1 to 30.

Most preferably, the present invention concerns the use of a compound comprising the general formula (I) wherein R1 is C substituted with alkyl or alkenyl which may be (C4-C_{Z}), Boron, or heterocyclic group forming a ring with R6 thus comprising a pyrrolidine further substituted one or more times with O, methyl, alicyclic groups attached to one another, these being saturated or unsaturated or a heterocyclic group comprising said Boron, N and O, and being further substituted at least once with O, methyl or C further substituted at least once with cycloalkenyl, cycloalkyl, heterocyclyl further substituted at least once with O, OH, methyl, alkenyl, further substituted with lower alkyl and cycloalkenyl; R2 is C substituted with OH or hydrogen; R3 is C substituted with OH, methoxy, lower alkyl (C1-C_{Z}) and may at any point form a ring with R4; R4 is C substituted with OH, methoxy, lower alkyl (C1-C_{Z}) and may at any point form a ring with R3; R5 is C substituted one or more times with hydrogen, OH, CHO or methyl which may form a ring with R6; and R6 is C substituted one or more times with hydrogen, CHO, lower alkyl (C1-C_{Z}) or methyl, which may form at least one bond with R5, or be forming a ring with R1, wherein the at least one bond between any of R1, R2, R3, R4, R5 and/or R6 may be single or double bonds and _{Y} is an integer of either 2 or 3, and _{Z} is an integer of either 5 or 12.

Certain molecular modifications are preferable especially regarding the classical vanilloid receptor agonist compounds, as they seem to ensure a more favorable degree of vanilloid receptor activation. These modifications include an unbranched alkyl side chain of a certain minimum length, as shorter chain analogues decrease in potency and thus are not capable of inducing hypothermia to a satisfactory degree. It is therefore preferable that the compounds of the present invention have relatively long alkyl or alkenyl chains (counted from R1 in the above formula) such as chains longer than 6 carbon atoms. Preferably the length of the alkyl/alkenyl chain is between 6 and 25 carbon atoms, most preferably between 7 and 18 carbon atoms, even more preferably between 8 and 9 carbon atoms.

In contrast hereto, substitution in the aromatic ring seems to abolish activity altogether and modification of many of the TRPV1 capsaicinoid and capsaicinoid-like agonists by halogenation of the aromatic ring tends to yield potent antagonists.

### Examples of Compounds

Examples of compounds specially relevant for the present invention include, but are not limited to classic vanilloid receptor agonists such as (names in parenthesis are alternative names): Capsaicin (C; 8-methyl-N-vallilyl-6-nonenamide), Dihydrocapsaicin (DHC), Nordihydro-capsaicin (NDHC), Homodihydro-capsaicin (HDHC), Homocapsaicin (HC), Olvanil (N-9-Z-octadecenoyl-vanillamide), Rinvanil (vanillamide of ricinoleic acid), Arvanil (N-vanillylarachidonamide), PhAR (phenylacetylrinvanil), Nuvanil, Farvanil (vanillamide of farnesic acid), Ac-Rinvanil, Retvanil (vanillamide of retinoic acid), Nonivamide, and Ervanil (vanillamide of erucic acid).

Examples of non-classical vanilloid receptor agonist compounds specially relevant for the present invention include, but are not limited to compounds such as (names in parenthesis are alternative names): Resiniferatoxin (RTX), Anandamide (arachidonyl ethanolamine), N-arachidonoyldopamine (NADA), N-arachidonoyl-L-serine (ARA-S), Arachidonyl-2-chloroethylamide, 2-aminoethoxydiphenyl borate (2-APB), Evodiamine, Propofol, Isovelleral, Scutigeral, 12-hydroperoxyeicosatetraenoic acid, Alpha-sanshool, Beta-sanshool, Gamma-sanshool, Delta-sanshool, Alpha-hydroxy-sanshool, Beta-hydroxy-sanshool, Piperine, Zingerone, and Bv8.

### Preferred Compounds

The compounds of the present invention may apart from inducing hypothermia induce secondary effects or have other characteristics. These may be related to the capsaicinoid nature of the compounds and may thus be more or less desirable. It is preferable that the compounds of the invention do not induce any blood-pressure modifying effects or induce hyperalgesic, nocifensive, plasma extravasation, peripheral vasodilatation, bronchoconstriction, bradycardia, or apnea effects among others. A potential analgetic effect may not be considered an adverse effect and may even, depending on the application of this invention, be desirable. Furthermore, a compound of the present invention may be hydrophilic or hydrophobic. To facilitate the administration of a compound according to the present invention it is preferable for a compound to be hydrophilic. A preferred compound is moreover metabolically stable.

A preferred compound of the present invention is a compound capable of binding the TRPV1 receptor, thereby inducing hypothermia in an individual to a temperature in the range of 36 to 32 degree Celsius, and where said compound is hydrophilic.

Examples of preferred or especially relevant compounds include: Capsaicin and compounds closely related to capsaicin such as: Dihydrocapsaicin (DHC), Nordihydro-capsaicin (NDHC), Homodihydro-capsaicin (HDHC), and Homocapsaicin other specially relevant compounds include resiniferatoxin and compounds closely related hereto.

### Antagonists

It is an object of the present invention to provide compounds that are capable of obviating the effect of the compounds that induce hypothermia. These compounds are herein termed antagonists and exert their antagonistic effect by blocking the ability of any of the vanilloid receptor agonists, capsaicinoids or capsaicinoid-like compounds herein described in binding to their receptors. The purpose of such an antagonist is to provide an additional safety mechanism whereby it is possible to stop the decline in core body temperature, stabilize the core body temperature and/or raise the core body temperature of an individual.

An embodiment of the present invention thus comprises the use of a compound according to any of the above for the preparation of a medicament for antagonizing the induction of hypothermia in an individual.

Examples of antagonists includes but is not limited to: 5-iodoresiniferatoxin, Aminoquinazoline (Aminoquinazoline 70), 6-iodo-nordihydrocapsaicin, IBTU (N-(4-chlorobenzyl)-N′-(4-hydroxy-3-iodo-5-methoxybenzyl)thiourea), KJM429 and JYL1421, A-425619, AMG9810, SB 366791, Adenosine and Capsazepine.

### Novel Use of Compounds

Vanilloid receptor agonists, capsaicinoids and capsaicinoid-like compounds have been used for a variety of purposes over time. It is an object of the present invention to provide a novel use of these compounds for the induction of hypothermia, especially for the induction of hypothermia in an individual suffering from ischemia or at risk of suffering from ischemia.

### Medicament

The induction of hypothermia by any of the herein described compounds is performed by preparing, producing and thus providing a medicament or pharmaceutical composition comprising at least one of said compounds. The medicament of the present invention is thus for the induction of hypothermia in an individual for the treatment and/or prophylaxis of ischemia in said individual.

### Pharmaceutical Composition

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier there for.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulphates, and sulfosuccinates, (c) non-ionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more non-ionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

A pharmaceutically acceptable salt means any salt of the compounds mentioned. In particular, it means a pharmaceutically acceptable acid addition salt. Pharmaceutically acceptable acid addition salts of the compounds include salts derived from non-toxic inorganic acids such as hydrochloric, nitric, phosphoric, sulphuric, hydrobromic, hydriodic, hydrofluoric, phosphorous and the like, as well as the salts derived from non-toxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulphate, pyrosulphate, bisulphate, sulphite, bisulphite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

pH

An aspect of the present invention regards the pH of the medicament. The pH of the medicament depends on the administration form, as the pH of the medicament preferably is suitable for the route of administration chosen. An embodiment of the present invention comprises a medicament wherein the pH of the composition is between pH 5 and pH 9, such as between pH 5.5 and 8.5 such as between pH 6 and 8, such as between pH 6.5 and 7.5. Most preferably the pH of the medicament is in accordance with the route of administration and the tissue to which the medicament is administered.

### Indications

The invention provides compounds for the production of a medicament for the treatment of ischemia in an individual. Ischemia may arise due to various circumstances and thus it is an object of the present invention to treat ischemia arising from a plurality of medical indications.

These indications include, but are not limited to, cardiovascular diseases such as myocardial infarction, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis, perinatal asphyxia and non-perinatal asphyxia as well as traumatic brain injury.

### Target Temperature and Peak Effect

The target temperature of the medicament is the core body temperature that can be reached upon administering the medicament according to the present invention as prescribed according to potency, dosage and so on. Various ranges and specific hypothermic core body temperatures that fall within the scope present invention are equal to the temperatures that the compound itself may induce as listed in the section on hypothermia.

An embodiment of the present invention is thus a medicament comprising a compound of the present invention capable of inducing hypothermia in the range of between 36 to 31 degree Celsius, and more preferably between 34 and 32 degree Celsius.

The compounds of this invention will often show a peak in hypothermia inducing effect 30 minutes to 120 minutes after administration, but may potentially peak sooner or later than described by this interval. The hypothermia inducing effect will frequently last from 1 hour to 12 hours, but may potentially last shorter or longer than described by this interval.

It is an object of the present invention to provide medicaments suitable for a quick induction of hypothermia which may be of long or short duration and medicaments suitable for a slow reduction in core body temperature which may be of long or short duration.

An embodiment of the present invention is thus a medicament capable of inducing hypothermia below 36° C., such as below 35.5° C., such as below 35° C., such as below 34.5° C., such as below 34.0° C. in the range of between 36 to 32 degree Celsius, and more preferably between 35 and 33 degree Celsius.

### Administration

The main routes of drug delivery, in the treatment method are intravenous, oral, and topical, as will be described below. Other drug-administration methods, such as subcutaneous injection or via inhalation, which are effective to deliver the drug to a target site or to introduce the drug into the bloodstream, are also contemplated.

The mucosal membrane to which the pharmaceutical preparation of the invention is administered may be any mucosal membrane of the individual to which the biologically active substance is to be given, e.g. in the nose, vagina, eye, mouth, genital tract, lungs, gastrointestinal tract, or rectum, preferably the mucosa of the nose, mouth or rectum.

Compounds of the invention may be administered parenterally, that is by intravenous, intramuscular, intraspinal, subcutaneous, intranasal, intrarectal, intravaginal or intraperitoneal administration. Appropriate dosage forms for such administration may be prepared by conventional techniques. The compounds may also be administered by inhalation, that is by intranasal and oral inhalation administration. Appropriate dosage forms for such administration, such as an aerosol formulation or a metered dose inhaler, may be prepared by conventional techniques.

The compounds according to the invention may be administered with at least one other compound. The compounds may be administered simultaneously, either as separate formulations or combined in a unit dosage form, or administered sequentially.

A preferred embodiment of the present invention is a medicament for administration by injection, suppository, oral administration, sublingual tablet or spray, cutaneous administration, or inhalation. More preferably the administration form is by injection, wherein the injection is intravenous, intramuscular, intraspinal, intraperitoneal, subcutaneous, a bolus or a continuous administration.

Some vanilloid receptor agonists may act as irritants under certain circumstances. Therefore, the compounds of the present invention, depending on the specific active substance, may preferably be administered as capsules rather than tablets or suppositories, and preferably as intravenous injections rather than subcutaneous or intramuscular injections.

### Individual

The individual that may benefit from the administration of a medicament as described herein may be an individual suffering from ischemia or at risk of suffering from ischemia. If the individual is at risk of suffering from ischemia the preferred administration form of the medicament may be suppository, oral administration or inhalation. Preferably, the individual is an individual suffering from ischemia. The preferred administration form for an individual suffering from ischemia is an injection, such as an intravenous, intramuscular, intraspinal, intraperitoneal or subcutaneous injection.

The individual may be any human being, male or female, infant or old. The ischemic condition to be treated or prevented in the individual may relate to the age of the individual, the general health of the individual and whether or not the individual has a prior history of suffering from diseases or disorders that may have or have induced ischemic conditions in the individual.

### Dosage

The dosage of the compound according to the invention depends on the compound in question; however, the amount of the compound is also closely related to the pharmaceutical composition of the medicament, any second compound of the medicament or any second active ingredient of the medicament.

For all methods of use disclosed herein for the compounds, the daily oral dosage regimen will preferably be from about 0.01 to about 80 mg/kg of total body weight. The daily parenteral dosage regimen will be from about 0.001 to about 80 mg/kg of total body weight.

For all methods of use disclosed herein for the compounds, the daily oral dosage regimen will preferably be from about 0.01 to about 80 mg/kg of total body weight. The daily parenteral dosage regimen will be from about 0.01 to about 2,400 mg/kg of total body weight, preferably, the dosage of the medicament according to the present invention is between 10 μg to 10 mg pr kg total body weight, such as between 100 μg to 1 mg pr kg total body weight, depending on the compound of choice. It has been found that vanilloids varies with respect to potency and affinity for the receptor as well as with respect to molecular weight.

For any other receptor agonist compound according to the invention the exact dosage may be calculated based on the porcine study model described in Example 14.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the individual that corresponds to a concentration of one or more compounds according to the invention. The effective level can also be defined as the amount needed pr kg body weight, in other words the concentration, required to reach the peak effect for a specific drug. Also, the effective level depends on the severity of the ischemic condition, such as total amount of tissue experiencing hyp- or anoxia, the duration of the ischemic condition, whether it is the first or a subsequent ischemic attack of the individual and so forth.

More preferably, the dosage of the medicament according to the present invention is between 10 μg to 80 mg pr kg total body weight, such as between 100 μg to 1 mg pr kg total body weight.

### Dosage Regime and Duration of Treatment

The medicament may be administered in any suitable dosage regime, suitable as to the potency of the compound/drug, the target temperature to be reached, the speed of action of the compound, the metabolic stability of the compound, the duration of the treatment and how often the medicament optimally is to be administered.

It is within the scope of the invention to provide a medicament to be administered at intervals of 30 minutes to 48 hours, such as intervals of 1 to 47 hours, 2 to 45 hours, 3 to 43 hours, 4 to 41 hours, 5 to 39 hours, 6 to 37 hours, 7 to 35 hours, 8 to 3 hours, 9 to 31 hours, 10 to 29 hours, 11 to 27 hours, 12 to 25 hours, 13 to 23 hours, 14 to 21 hours, 15 to 19 hours 16 to 18 hours. It is also within the scope of the present invention to provide a medicament to be administered at intervals of 30 minutes to 24 hours, such as 1 to 23 hours, 2 to 22 hours, 3 to 20 hours, 4 to 18 hours, 5 to 16 hours, 6 to 14 hours, 7 to 12 hours or 8 to 10 hours. Preferably, the administration occurs at intervals of 1 to 6 hours, such as 2 to 5 hours, 3 to 4 hours.

The optimal administration interval depends on the duration of the hypothermic treatment. The duration of the treatment depends among other things on the severity of the ischemic condition. It is within the scope of the present invention to provide medicaments for the induction of hypothermia wherein the duration of the treatment is from 6 to 72 hours, such as from 7 to 69 hours, such as from 8 to 66 hours, 9 to 63 hours, 10 to 60 hours, 11 to 57 hours, 12 to 54 hours, 13 to 51 hours, 14 to 48 hours, 15 to 45 hours, 16 to 42 hours, 17 to 39 hours, 18 to 36 hours, 1 to 35 hours, 20 to 32 hours, 21 to 29 hours, 22 to 26 hours 23 to 25 hours. Preferably, the duration of the treatment is between 6 and 48 hours, more preferably between 6 and 24 hours.

### Multiple Compound Medicaments

An object of the present invention is to provide compounds capable of inducing hypothermia in an individual. The induction of hypothermia depends on the characteristics of the compounds and these characteristics may be to reach different target temperatures or different ranges of target temperatures, reaching the target temperatures at various speeds (varying peak effect times), the duration of the induced hypothermia, the lifetime of the active compound and so on. It is therefore an object of the present invention to provide medicaments comprising more than one compound, such as at least two, at least three or at least four compounds as herein described.

The medicament may thus comprise compounds of the present invention wherein at least one compound induces hypothermia rapidly, or alternatively wherein at least one compound induces hypothermia slowly. The medicament may furthermore comprise at least one compound that induces hypothermia over an extended time period, alternatively wherein at least one compound induces hypothermia for a short time period.

### Second Active Ingredient

An embodiment of the present invention is a pharmaceutical composition comprising a compound as herein described and furthermore comprising a second active ingredient. The second active ingredient may increase the hypothermic effect of the compound of the invention, or may have an alternative medical effect such as inducing pain relief or vasodilation.

The second active ingredient may thus be selected from the non-limiting group of: cannabinoids, neurotensins, analgesics, opiods, GABAs and adrenergic antagonists.

Examples of these include, but are not limited to: cannabinoids such as anandamide (anandamide is an agonist of both the cannabinoid receptor CB1 and the vanilloid receptor VR1 and may therefore be referred to both as a cannabinoid and a capsaicinoid/vanilloid receptor agonist), delta-9-THC, delta-8-THC, cannabidiol, HU210, BAY 38-7271, WIN 55,212 and CP55940 and the phosphate derivates of these, and neurotensin analogues KK13 and KK14.

### Kit of Parts

Another embodiment of the present invention comprises a kit of parts, wherein the kit includes at least one pharmaceutical composition according to any of the above, a means for administering said vaccine and the instruction(s) on how to do so. It is within the scope of the present invention to include multiple dosages of the same composition or several different compositions. In a preferred embodiment the kit of parts further comprises a second active ingredient.

### EXAMPLES

### Example 1

### Cardiac Arrest

A 57-year-old woman is taken care of by the ambulance staff that finds her in ventricular fibrillation approximately 5 minutes after having collapsed without warning. The patient is immediately defibrillated and spontaneous circulation and ventilations occurred. On arrival to the hospital, 21 minutes after having collapsed, the patient has a palpable pulse. Staff at the emergency room has been alerted in advance. The patient is evaluated and the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 2

### Cardiac Arrest

A 22-year-old male athlete collapses when doing sports. Medical staff finds ventricular fibrillation/ventricular tachycardia approximately 6 minutes after collapse. He is successfully defibrillated and the patient is rushed to the hospital. In the ambulance, the physician present in the ambulance team decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 3

### Cardiac Arrest

A 66-year-old man is undergoing elective heart surgery. He suffers from irregular cardiac rhythm during the procedure and goes into cardiac arrest with much impaired circulation for 6 minutes after which the surgical team manages to restore circulation. Subsequent to restoration of circulation, the surgeon in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 4

### Cardiac Arrest

An electrician accidentally receives a high-voltage jolt and immediately slips into a coma. The company physician manages to resuscitate the cardiac arrest patient after just 7 minutes. The patient is rushed to hospital where the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 5

### Perinatal Asphyxia

A newborn baby suffers cerebral ischemia during delivery as the umbilical cord gets wrapped around his neck. The APGAR score 10 minutes after delivery is 6. The patient is evaluated and the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 6

### Asphyxia

A 9-year-old boy is in a coma when he is rescued out of a burning house. CRP is commenced on the spot but he is not awake when the ambulance arrives at hospital after a 15 minutes drive. The patient is evaluated and the physician in charge decides that the patient shall receive hypothermia therapy immediately to minimize the risk of damage to the brain and other tissues. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 7

### Stroke

A 78-year-old woman is admitted to hospital 50 minutes after experiencing sudden onset lack of mobility of the left arm and leg and as well and difficulties speaking. The patient is diagnosed with a stroke. The patient is evaluated and the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 8

### Stroke

A 29-year-old man suffers continuous pains in the back of his head for two weeks after which he suddenly collapses and slips into a coma. The patient is diagnosed with a stroke. The patient is evaluated and the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 9

### Myocardial Infarction

A 55-year-old man is admitted to hospital 18 minutes after experiencing nausea, shortness of breath, and severe chest pains. The patient, who is suffering myocardial infarction, is evaluated by the physician in charge who decides that the patient shall receive hypothermia therapy immediately to minimize the risk of damage to the heart and other tissues. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 10

### Traumatic Brain Injury

A 41-year-old man receives a severe blow to the head from a falling brick on a construction site. The patient is still unconscious when he 24 minutes after the trauma is brought to the Emergency Room. The patient is evaluated and the physician in charge decides that the patient shall receive hypothermia therapy immediately to minimize the risk of damage to the brain. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 11

### Pulmonary Embolism

A 60-year-old woman is feeling rather well two days after major surgery when she suddenly slips into a coma. Following acute surgery, brought on by a pulmonary embolism, she is stabilized. The patient is evaluated and the physician in charge decides that the patient shall receive hypothermia therapy immediately to minimize the risk of damage to the brain and other tissues. A subcutaneous or intravenous bolus injection of a compound of this invention is administered. The dose will likely be in the interval 0.01 mg/kg to 80 mg/kg.

The purpose of hypothermia therapy is to lower the patient's core body temperature to 32-34 degrees Celsius for 12-24 hours (current American Heart Association recommendation). Depending on the individual's response to the medication 1-8 additional bolus injections may be required.

At the hospital, concurrent treatments and examinations are not influenced by the administration of the hypothermia-inducing drug. Such other treatments and examinations proceed uninterrupted.

### Example 12

### Modified Pyrogenicity Test on Rabbits

Each active substance in a low, medium and high dose will be screened initially on 3 rabbits. Temperature, blood pressure, pulse will be measured for 72 hours following administration of active substances. These parameters will be recorded as follows:

Continuous recording (time=0-3 h),
every 30 minutes (time=3-6 h),
every 1 hour (time=6-12 h),
every 2 hours (time=12-24 h),
every 6 hours (time=24-48 h),
every 12 hours (time=48-72 h)

These are the specifics of the initial screening:

Type of rabbits: New Zealand White, Charles River

### Weight: >1.5 kg

### Gender: Female

Room temperature: 21° C. (+/−1° C.)
Relative humidity: 55% (+/−5%)
Type of temperature probe: PC Based pyrogen testing system, Ellab APT 91
Cage type: Pro Plast Noryl, 2475 cm2
Number of rabbits in a cage: 1
Water and food access: Ad lib in cage.
12-hour light period: Yes

### Example 13

### Receptor Studies

Modulation of TRPV1 activity will be assessed in vitro by measuring vanilloid receptor agonist induced Ca2+ flux using FLIPR and HEK293 cells stably expressing recombinant human and rat TRPV1 (hTRPV1-HEK293 and rTRPV1-HEK293, respectively). Intracellular Ca2+ levels will be measured in TRPV1-expressing cells during exposure to compounds. A concentration dependent increase in Ca2+ influx will be observed using both human and rat cell lines. The efficacy of agonists will be estimated by comparing it to the maximum response induced by capsaicin.

### Example 14

### Porcine Study Model

In order to evaluate an effective hypothermic dose of a receptor agonist compound according to the invention, the compound may be tested in the porcine study model. The porcine model is used because the body weight of the pigs is comparable to the body weight of humans. The efficacy of a compound tested in the porcine model may be correlated with the efficacy of the vanilloid compound tested in the same porcine study model.

### Study Subjects

The evaluation is carried out on “dansk landrace” pigs with a body weight of 70-90 kilo. The pigs will not be sedated; they will be fed twice a day: and they will be subjected to a day cycle consisting of 12 hours of light followed by 12 hours of dark.

### Drug Administration

The vanilloid compound investigated is administered i.v. as bolus injections and may consist of 1 solitary injection, alternatively 2-4 repeated injections within a timeframe of 24 hours from the initial injection.

Generally 4 different doses plus vehicle are tested producing varying degrees of hypothermic responses.

### Hypothermic Effect

The primary effect evaluated is hypothermia. Temperature is measured using a temperature probe that is surgically positioned in a femoral artery two weeks prior to commencement of the study. The probe is connected to telemetry equipment (e.g. implanted telemetry from Data Sciences International) ensuring the required readouts.

Temperature is measured every 15 minutes from 1 hour prior to drug administration to 12 hours after drug administration, and every 30 minutes subsequently until 24 hours after drug administration. Temperature measurement will be conducted via a permanent femoral artery temperature probe (telemetry).

The minimum temperature as well as a graph of the temperature at each point of measurement is recorded for each dose of vanilloid.

### Other Effects

Blood pressure, heart rate and ECG will be registered every 15 minutes from 1 hour prior to drug administration to 12 hours after drug administration, and every 30 minutes subsequently until 24 hours after drug administration.

### REFERENCES

(1) Busto R, Dietrich W D, Globus M Y, Valdes I, Scheinberg P, Ginsberg M D: Small differences in intraischemic brain temperature critically determine the extent of ischemic neuronal injury. J Cereb Blood Flow Metab 1987; 7 (6):729-738.
(2) Barone F C, Feuerstein G Z, White R F: Brain cooling during transient focal ischemia provides complete neuroprotection. Neurosci Biobehav Rev 1997; 21 (1):31-44.
(3) Onesti S T, Baker C J, Sun P P, Solomon R A: Transient hypothermia reduces focal ischemic brain injury in the rat. Neurosurgery 1991; 29 (3):369-373.
(4) Coimbra C, Wieloch T: Moderate hypothermia mitigates neuronal damage in the rat brain when initiated several hours following transient cerebral ischemia. Acta Neuropathol (Berl) 1994; 87 (4):325-331.
(5) Zhang Y, Wong K C, Zhang Z: The effect of intraischemic mild hypothermia on focal cerebral ischemia/reperfusion injury. Acta Anaesthesiol Sin 2001; 39 (2):65-69.
(6) Yamashita K, Eguchi Y, Kajiwara K, Ito H: Mild hypothermia ameliorates ubiquitin synthesis and prevents delayed neuronal death in the gerbil hippocampus. Stroke 1991; 22 (12):1574-1581.
(7) Ooboshi H, Ibayashi S, Takano K, Sadoshima S, Kondo A, Uchimura H, Fujishima M: Hypothermia inhibits ischemia-induced efflux of amino acids and neuronal damage in the hippocampus of aged rats. Brain Res 2000; 884 (1-2):23-30.
(8) Colbourne F, Corbett D, Zhao Z, Yang J, Buchan A M: Prolonged but delayed postischemic hypothermia: a long-term outcome study in the rat middle cerebral artery occlusion model. J Cereb Blood Flow Metab 2000; 20 (12):1702-1708.
(9) Kawai N, Okauchi M, Morisaki K, Nagao S: Effects of delayed intraischemic and postischemic hypothermia on a focal model of transient cerebral ischemia in rats. Stroke 2000; 31 (8):1982-1989.
(10) Maier C M, Sun G H, Kunis D, Yenari M A, Steinberg G K: Delayed induction and long-term effects of mild hypothermia in a focal model of transient cerebral ischemia: neurological outcome and infarct size. J Neurosurg 2001; 94 (1):90-96.
(11) Maier C M, Ahern K, Cheng M L, Lee J E, Yenari M A, Steinberg G K: Optimal depth and duration of mild hypothermia in a focal model of transient cerebral ischemia: effects on neurologic outcome, infarct size, apoptosis, and inflammation. Stroke 1998; 29 (10):2171-2180.
(12) Mild therapeutic hypothermia to improve the neurologic outcome after cardiac arrest: N Engl J Med 2002; 346 (8):549-556.
(13) Bernard S A, Gray T W, Buist M D, Jones B M, Silvester W, Gutteridge G, Smith K: Treatment of comatose survivors of out-of-hospital cardiac arrest with induced hypothermia. N Engl J Med 2002; 346 (8):557-563.
(14) Bernard S: Therapeutic hypothermia after cardiac arrest: now a standard of care. Crit. Care Med 2006; 34 (3):923-924.
(15) Dietrich W D, Halley M, Valdes 1, Busto R: Interrelationships between increased vascular permeability and acute neuronal damage following temperature-controlled brain ischemia in rats. Acta Neuropathol (Berl) 1991; 81 (6):615-625.
(16) Karibe H, Zarow G J, Graham S H, Weinstein P R: Mild intraischemic hypothermia reduces postischemic hyperperfusion, delayed postischemic hypoperfusion, blood-brain barrier disruption, brain edema, and neuronal damage volume after temporary focal cerebral ischemia in rats. J Cereb Blood Flow Metab 1994; 14 (4):620-627.
(17) Huang Z G, Xue D, Preston E, Karbalai H, Buchan A M: Biphasic opening of the blood-brain barrier following transient focal ischemia: effects of hypothermia. Can J Neurol Sci 1999; 26 (4):298-304.
(18) Bagenholm R, Nilsson U A, Kjellmer I: Formation of free radicals in hypoxic ischemic brain damage in the neonatal rat, assessed by an endogenous spin trap and lipid peroxidation. Brain Res 1997; 773 (1-2):132-138.
(19) Kil H Y, Zhang J, Piantadosi C A: Brain temperature alters hydroxyl radical production during cerebral ischemia/reperfusion in rats. J Cereb Blood Flow Metab 1996; 16 (1):100-106.
(20) Kumura E, Yoshimine T, Takaoka M, Hayakawa T, Shiga T, Kosaka H: Hypothermia suppresses nitric oxide elevation during reperfusion after focal cerebral ischemia in rats. Neurosci Lett 1996; 220 (1):45-48.
(21) Baker A J, Zornow M H, Grafe M R, Scheller M S, Skilling S R, Smullin D H, Larson M: Hypothermia prevents ischemia-induced increases in hippocampal glycine concentrations in rabbits. Stroke 1991; 22 (5):666-673.
(22) Takagi K, Ginsberg M D, Globus M Y, Dietrich W D, Martinez E, Kraydieh S, Busto R: Changes in amino acid neurotransmitters and cerebral blood flow in the ischemic penumbral region following middle cerebral artery occlusion in the rat: correlation with histopathology. J Cereb Blood Flow Metab 1993; 13 (4):575-585.
(23) Nakashima K, Todd M M: Effects of hypothermia on the rate of excitatory amino acid release after ischemic depolarization. Stroke 1996; 27 (5):913-918.
(24) Maier C M, Sun G H, Cheng D, Yenari M A, Chan P H, Steinberg G K: Effects of mild hypothermia on superoxide anion production, superoxide dismutase expression, and activity following transient focal cerebral ischemia. Neurobiol Dis 2002; 11 (1):28-42.
(25) Pulsinelli W: Pathophysiology of acute ischaemic stroke. Lancet 1992; 339 (8792):533-536.
(26) Eguchi Y, Yamashita K, Iwamoto T, Ito H: Effects of brain temperature on calmodulin and microtubule-associated protein 2 immunoreactivity in the gerbil hippocampus following transient forebrain ischemia. J Neurotrauma 1997; 14 (2):109-118.
(27) Sick T J, Tang R, Perez-Pinzon M A: Cerebral blood flow does not mediate the effect of brain temperature on recovery of extracellular potassium ion activity after transient focal ischemia in the rat. Brain Res 1999; 821 (2):400-406.
(28) Hu B R, Kamme F, Wieloch T: Alterations of Ca2+/calmodulin-dependent protein kinase 11 and its messenger RNA in the rat hippocampus following normo- and hypothermic ischemia. Neuroscience 1995; 68 (4):1003-1016.
(29) Busto R, Globus M Y, Neary J T, Ginsberg M D: Regional alterations of protein kinase C activity following transient cerebral ischemia: effects of intraischemic brain temperature modulation. J Neurochem 1994; 63 (3):1095-1103.
(30) Ishikawa M, Sekizuka E, Sato S, Yamaguchi N, Inamasu J, Bertalanffy H, Kawase T, Iadecola C: Effects of moderate hypothermia on leukocyte-endothelium interaction in the rat pial microvasculature after transient middle cerebral artery occlusion. Stroke 1999; 30 (8):1679-1686.
(31) Inamasu J, Suga S, Sato S, Horiguchi T, Akaji K, Mayanagi K, Kawase T: Postischemic hypothermia delayed neutrophil accumulation and microglial activation following transient focal ischemia in rats. J Neuroimmunol 2000; 109 (2):66-74.
(32) Hara A, Niwa M, Iwai T, Yano H, Bunai Y, Uematsu T, Yoshimi N, Mori H: Increase of fragmented DNA transport in apical dendrites of gerbil CA1 pyramidal neurons following transient forebrain ischemia by mild hypothermia. Neurosci Lett 2000; 280 (1):73-77.
(33) Xu L, Yenari M A, Steinberg G K, Giffard R G: Mild hypothermia reduces apoptosis of mouse neurons in vitro early in the cascade. J Cereb Blood Flow Metab 2002; 22 (1):21-28.
(34) Zhang Z, Sobel R A, Cheng D, Steinberg G K, Yenari M A: Mild hypothermia increases Bcl-2 protein expression following global cerebral ischemia. Brain Res Mol Brain Res 2001; 95 (1-2):75-85.
(35) Niwa M, Hara A, Iwai T, Nakashima M, Yano H, Yoshimi N, Mori H, Uematsu T: Relationship between magnitude of hypothermia during ischemia and preventive effect against post-ischemic DNA fragmentation in the gerbil hippocampus. Brain Res 1998; 794 (2):338-342.
(36) Inamasu J, Suga S, Sato S, Horiguchi T, Akaji K, Mayanagi K, Kawase T: Postischemic hypothermia attenuates apoptotic cell death in transient focal ischemia in rats. Acta Neurochir Suppl 2000; 76:525-527.
(37) Silva C, Silva J, Ribeiro M J, Avelino A, Cruz F: Urodynamic effect of intravesical resiniferatoxin in patients with neurogenic detrusor overactivity of spinal origin: results of a double-blind randomized placebo-controlled trial. Eur Urol 2005; 48 (4):650-655.
(38) Payne C K, Mosbaugh P G, Forrest J B, Evans R J, Whitmore K E, Antoci J P, Perez-Marrero R, Jacoby K, Diokno A C, O'Reilly K J, Griebling T L, Vasavada S P, Yu A S, Frumkin L R: Intravesical resiniferatoxin for the treatment of interstitial cystitis: a randomized, double-blind, placebo controlled trial. J Urol 2005; 173 (5):1590-1594.
(39) Kim J H, Rivas D A, Shenot P J, Green B, Kennelly M, Erickson J R, O'Leary M, Yoshimura N, Chancellor M B: Intravesical resiniferatoxin for refractory detrusor hyperreflexia: a multicenter, blinded, randomized, placebo-controlled trial. J Spinal Cord Med 2003; 26 (4):358-363.
(40) Hayes A G, Oxford A, Reynolds M, Shingler A H, Skingle M, Smith C, Tyers M B: The effects of a series of capsaicin analogues on nociception and body temperature in the rat. Life Sci 1984; 34 (13):1241-1248.
(41) Woods A J, Stock M J, Gupta A N, Wong T T, Andrews P L: Thermoregulatory effects of resiniferatoxin in the rat. Eur J Pharmacol 1994; 264 (2):125-133.
(42) Miller M S, Brendel K, Buck S H, Burks T F: Dihydrocapsaicin-induced hypothermia and substance P depletion. Eur J Pharmacol 1982; 83 (3-4):289-292.
(43) Ding Z, Cowan A, Rawls S M: Capsaicin evokes hypothermia independent of cannabinoid CB1 and CB2 receptors. Brain Res 2005; 1065 (1-2):147-151.
(44) Jakab B, Helyes Z, Varga A, Bolcskei K, Szabo A, Sandor K, Elekes K, Borzsei R, Keszthelyi D, Pinter E, Petho G, Nemeth J, Szolcsanyi J: Pharmacological characterization of the TRPV1 receptor antagonist JYL1421 (SC0030) in vitro and in vivo in the rat. Eur J Pharmacol 2005; 517 (1-2):35-44.
(45) Swanson D M, Dubin A E, Shah C, Nasser N, Chang L, Dax S L, Jetter M, Breitenbucher J G, Liu C, Mazur C, Lord B, Gonzales L, Hoey K, Rizzolio M, Bogenstaetter M, Codd E E, Lee D H, Zhang S P, Chaplan S R, Carruthers N I: Identification and biological evaluation of 4-(3-trifluoromethylpyridin-2-yl)piperazine-1-carboxylic acid (5-trifluoromethylpyridin-2-yl)amide, a high affinity TRPV1 (VR1) vanilloid receptor antagonist. J Med Chem 2005; 48 (6):1857-1872.

1.-44. (canceled)

45. A vanilloid receptor agonist for induction of hypothermia in a human being, wherein the compound is a classic or non-classic vanilloid receptor agonist of the general formula (I): 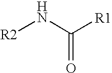
wherein R1 and R2 are chemical moieties or chemical bonds.

46. A method for the use of a compound as defined in claim 45, wherein R1 is selected from the group of: C, S, N, O, optionally substituted with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, ethyl, dimethyl, or may be further substituted one or more times with C, S, N, O, P, OH, methoxy, ethoxy, acetyl, alkoxy, alkyl, alkenyl, alkynyl, sulfonyl or phenyl any of which may or may not be branched or comprise substituents such as hydrogen, methyl, ethyl, alkyl, alkenyl, alkynyl, alkoxy, phosphate, cycloalkyl, heterocycloalkyl, cycloalkenyl, dimethyl or phenyl and preferably is C substituted with C, O, P, H, OH, phosphate, alkyl, alkenyl, alkynyl any of which may be (C1-CV), or phenyl, any of which may be substituted with O, OH, methyl, dimethyl, alkyl, alkenyl, cycloalkyl, heterocycloalkyl, cycloalkenyl, phenyl, methoxy, ethoxy, alkoxy or phosphate, any of which may be further substituted with methyl, ethyl or phenyl and more preferably is C substituted with alkyl, alkenyl, any of which may be (C4-CW) any of which may be further substituted with O, OH, methoxy, ethoxy or methyl any of which may be further substituted with methyl, ethyl, or phenyl, wherein V is an integer of from 1 to 30 and W is an integer of from 5 to 18.

47. A method for the use of a compound as defined in claim 45, wherein R2 is selected from the group of: C, S, N, O, optionally substituted one or more times with C, S, N, O, P, OH, hydrogen, alkoxy, alkyl, alkenyl, alkynyl, phenyl, diphenyl, benzyl, amine (NH), halogen, substituted lower alkyl, alkenyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl or phosphate, optionally further substituted one or more times with C, S, N, O, P, OH, H, COOH, phenyl, amine (NH), halogen, alkoxy, substituted lower alkyl, alkyl or alkenyl such as (C1-CV), cycloalkenyl, sulphate, phosphate, aryl, heterocyclyl, heteroaryl, aryl-(C1-4)-alkyl, heteroaryl-(C1-4)-alkyl, heterocyclyl-(C1-4)-alkyl, cycloalkylalkyl, dicycloalkyl, tricycloalkyl, cycloalkenyl, alkoxy, carboxy, halogen, cyano, amino, nitro, or alcohol, any of which may be further substituted one or more times with OH, methyl, dimethyl, alkyl or alkenyl such as (C1-CV), alkoxy, phenyl, sulphate, phosphate, aryl, heteroaryl, carboxy, amino, nitro, alcohol or halogen and preferably is C, substituted one or more times with C, N, O, P, OH, hydrogen, alkoxy, alkyl, alkenyl, amine (NH), halogen, substituted lower alkyl, alkenyl, aryl, cycloalkyl, cycloalkenyl or phosphate, optionally further substituted one or more times with C, N, O, OH, COOH, hydrogen, amine (NH), halogen, alkoxy, substituted lower alkyl or alkenyl such as (C1-CX), phosphate, cycloalkenyl, alkoxy, carboxy or halogen, any of which may be further substituted one or more times with OH, methyl, dimethyl, alkyl or alkenyl such as (C1-CX), alkoxy, phenyl, sulphate, phosphate, carboxy or halogen and more preferably is C substituted with either: cycloalkenyl further substituted at least twice with any of OH or methoxy, or: lower alkyl such as (C1-C2) at least once and further substituted one or more times with OH, COOH, Chloride, methyl or cycloalkenyl, optionally further substituted one or more times with OH or methoxy and wherein V is an integer of from 1 to 30 and X is an integer of from 1 to 5.

48. A method for the use of a compound as defined in claim 45, wherein R1 is C substituted with C, O, P, H, OH, phosphate, alkyl, alkenyl, alkynyl any of which may be (C1-CV), or phenyl, any of which may be further substituted one or more times with O, OH, methyl, dimethyl, alkyl, alkenyl, cycloalkyl, heterocycloalkyl, cycloalkenyl, acetyl, phenyl, methoxy, ethoxy, alkoxy or phosphate, any of which may be further substituted with methyl, ethyl or phenyl and R2 is C, substituted one or more times with C, N, O, P, OH, hydrogen, alkoxy, alkyl, alkenyl, amine (NH), halogen, substituted lower alkyl, alkenyl, aryl, cycloalkyl, cycloalkenyl or phosphate, optionally further substituted one or more times with C, N, O, OH, COOH, hydrogen, amine (NH), halogen, alkoxy, substituted lower alkyl or alkenyl such as (C1-CX), phosphate, cycloalkenyl, alkoxy, carboxy or halogen, any of which may be further substituted one or more times with OH, methyl, dimethyl, alkyl or alkenyl such as (C1-CX), alkoxy, phenyl, sulphate, phosphate, carboxy or halogen and wherein V is an integer of from 1 to 30 wherein X is an integer of from 1 to 5.

49. A method for the use of a compound as defined in claim 45, wherein R1 is C substituted with alkyl, alkenyl, any of which may be (C4-CW) any of which may be further substituted one or more times with O, OH, acetyl, methoxy, ethoxy or methyl any of which may be further substituted one or more times with methyl, ethyl, cycloalkenyl or phenyl and W is an integer of from 5 to 18, and R2 is C substituted with either: cycloalkenyl further substituted at least twice with any of OH or methoxy, or: lower alkyl such as (C1-C2) at least once and further substituted one or more times with OH, COOH, Chloride, methyl or cycloalkenyl, optionally further substituted one or more times with OH or methoxy.

50. A method for the use of a compound as defined in claim 45, wherein the compound is dihydrocapsaicin.

51. A method for the use of a compound as defined in claim 45, wherein the induction of hypothermia is for the treatment of ischemia in said human being.

52. A method for the use as defined in claim 45, wherein the vanilloid receptor is TRPV1-6 and/or a receptor associated herewith.

53. A method for the use as defined in claim 45, wherein the vanilloid receptor is TRPV1.

54. A method for the use of a compound as defined in claim 45, which is hydrophilic.

55. A method for the use as defined in claim 45 of a medicament according to the induction of hypothermia in an individual suffering from or at risk of suffering from ischemia.

56. A method for the use as defined in claim 55 of a medicament for prophylaxis and/or treatment of ischemia in connection with cardiovascular diseases, asphyxia and/or traumatic brain injuries.

57. A method for the use as defined in claim 56, wherein the ischemia is due to cardiovascular diseases such as: myocardial infarction, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

58. A method for the use as defined in claim 56, wherein the ischemia is due to asphyxia such as: perinatal asphyxia and/or non-perinatal asphyxia.

59. A medicament comprising a compound according to claim 45 capable of inducing hypothermia in a human being.

60. The medicament according to claim 59, for prophylactic and/or therapeutic applications.

61. The medicament according to claim 59 for therapeutic applications.

62. The medicament according to claim 59, wherein the medicament induces hypothermia of between 32 and 36 degree Celsius.

63. The medicament according to claim 59, comprising at least two compounds according to claim 1.

64. The medicament according to claim 63, wherein at least one compound induces hypothermia rapidly.

65. The medicament according to claim 63, wherein at least one compound induces hypothermia slowly.

66. The medicament according to claim 59, comprising a second active ingredient.

67. The medicament according to claim 66 wherein the second active ingredient is selected from the group of: cannabinoids, neurotensins, analgesics, opiods, GABAs and adrenergic antagonists.

68. The medicament according to claim 59, comprising a pharmaceutically acceptable carrier.

69. The medicament according to claim 59, wherein the pH of the composition is between pH 5 and pH 9.

70. The medicament according to claim 59, for administration by injection, suppository, oral administration, sublingual tablet or spray, cutaneous administration, or inhalation.

71. The medicament according to claim 70, wherein the injection is intravenous, intramuscular, intraspinal, intraperitoneal, subcutaneous, a bolus or a continuous administration.

72. The medicament according to claim 59, wherein administration occurs at intervals of 30 minutes to 48 hours.

73. The medicament according to claim 59, wherein administration occurs at intervals of 1 to 6 hours.

74. The medicament according to claim 59, wherein the duration of the treatment is from 6 to 72 hours.

75. The medicament according to claim 59, wherein the dosage of the medicament is between 10 μg to 80 mg pr kg body mass.

76. A kit of parts comprising the medicament as defined in claim 59.

77. A method for treating ischemia in a human being in need thereof comprising administering to said human being an effective amount of a vanilloid receptor agonist capable of inducing hypothermia, said compound being as defined in claim 45.
